(19) United States
(12) Patent Application Publication
(10) Pub. No.: US 2006/0147998 A1
(43) Pub. Date: Jul. 6, 2006

(54) Method for diagnosing and monitoring critically ill patients

(76) Inventors: Gregory Ray Jones, Hillsborough, NC (US); Larisa Borzhemskaya, Youngsville, NC (US); Donald G. Hanson, Raleigh, NC (US); Rafael Angel Estevez, Raleigh, NC (US); Mark S. Wilson, Hillsborough, NC (US); John Glenn Link, Durham, NC (US); Bryan Barnes, Hull, MA (US)

Correspondence Address: BIOMERIEUX, INC., PATENT DEPARTMENT, 100 RODOLPHE STREET, DURHAM, NC, 27712, US

(21) Appl. No.: 11/290,722

(22) Filed: Nov. 30, 2005

Related U.S. Application Data
(60) Provisional application No. 60/632,431, filed on Dec. 1, 2004.

(51) Int. Cl. G01N 33/53

(52) U.S. Cl. 435/7.1

(57) ABSTRACT

Provided is an improved method of diagnosing and monitoring hemostatic dysfunction, sepsis-related morbidity or severe infection by improving detection of an in vitro complex formed by lipoprotein and C-reactive protein with the utilization of an effective amount of a surface active agent in the reagent.

### FIELD OF INVENTION

This invention relates to an improved method of diagnosing and monitoring severe infection and hemostatic dysfunction by enhancing the detection of a complex of C-reactive protein and lipoprotein.

### BACKGROUND OF THE INVENTION

There is an increasing recognition of common and overlapping pathophysiological pathways that link inflammation and coagulation. Early detection and enhanced monitoring of hemostatic dysfunction, particularly coagulation errors associated with inflammation and severe infection, can be essential in successfully treating the underlying condition. Severe infection may include a diagnosis of sepsis, sepsis-related morbidity and septic shock as well as disseminated intravascular coagulation (DIC). As described in WO 01/96864, one marker useful for early detection of hemostatic dysfunction (errors in coagulation) is a divalent cation dependent complex between C-reactive protein (CRP) and lipoprotein, commonly referred to as an LC-CRP complex.

The LC-CRP complex associated with critically ill patients may be detected by a number of means including time dependent measurement profiles of coagulation screening assays as described Toh et al. WO 00/46603, hereby incorporated by reference. As described by Toh, et al., for those patients with hemostatic dysfunction such as DIC, the LC-CRP complex precipitate formed upon addition to calcium to a common clotting assay, the activated partial thromboplastin time (“APTT”) assay. This precipitate results in a change of turbidity that causes a decrease in plasma light transmittance before clot formation (pre-coagulation), now commonly referred to as a biphasic waveform. The detection of biphasic waveforms on automated coagulation instruments provides one convenient method of detecting and monitoring the LC-CRP complex to diagnose and monitor patients. The LC-CRP complex may also be detected by different types of clotting assays other than APTT, latex agglutination or gold sol assays or an immunoassay specific to the LC-CRP complex such as described in WO 01/96864.

Methods to improve the specificity and sensitivity in detecting the LC-CRP complex would enhance the capability of the complex to be used as a broader spectrum biomarker for hemostatic dysfunction, sepsis-related morbidity and severe infection. Such improved methods are of importance for a clinical diagnostic as well as a therapeutic monitoring utility.

### SUMMARY OF THE INVENTION

It has been discovered that diagnosis and monitoring of hemostatic dysfunction, sepsis-related morbidity or severe infection associated with the LC-CRP complex of critically ill patients may be improved with a method comprising (a) obtaining a patient blood sample; (b) combining said sample with a divalent cation component and an effective amount of surface-active agent to form a reaction mixture; and (c) examining said reaction mixture to determine whether an LC-CRP complex is formed to diagnose or monitor patients having hemostatic dysfunction, sepsis-related morbidity or severe infection. The method has been found to improve precipitation and/or detection of the in vitro LC-CRP complex, particularly when the patient's lipoprotein profile becomes very low density lipoprotein (VLDL) poor and low-density lipoprotein (LDL) and/or an intermediate density lipoprotein (IDL) enriched. Also provided with the invention is a method for determining the increased likelihood of organ system failure or mortality of patients having an LC-CRP complex formed ex vivo utilizing steps (a)-(d) above.

In yet another aspect of the invention, a method for diagnosing hemostatic dysfunction, sepsis-related morbidity and severe infection is provided, said method comprising (a) obtaining a patient sample; (b) combining said sample with reagents comprising a calcium component and surface active reagent to form a reaction mixture; (c) subjecting said reaction mixture to a waveform analysis to obtain a waveform result; (e) comparing said waveform result with known samples in a model to diagnosis of severe infection, sepsis-related morbidity or hemostatic dysfunction. Still further, the detection of an LC-CRP complex is improved with an instrument comprising a means for measuring or monitoring the turbidity of an LC-CRP formed by the methods described above.

### BRIEF DESCRIPTION OF THE DRAWINGS

FIG. 1 illustrates the density and diameter of lipoprotein subclasses HDL (high density lipoprotein), LDL (low density lipoprotein); Lp(a) (lipoprotein (a)), IDL (intermediate density lipoprotein); and VLDL (very low density lipoprotein).

FIG. 2 illustrates the optical detection of LDL concentration (mg/dL) when differing concentrations of an anionic surfactant (0% to 0.003% w/v) are added to an LC-CRP-CaCl₂ reagent.

FIG. 3 illustrates the optical detection of VLDL concentration (mg/dL) when differing concentrations of an anionic surfactant (0% to 0.003% w/v) are added to an LC-CRP-CaCl₂ reagent.

FIG. 4 illustrates the optical detection of all fractions of beta lipoproteins present in normal pooled plasma (NPP) (mg/dL) when differing concentrations of an anionic surfactant (0% to 0.003 w/v) are added to an LC-CRP-CaCl₂ reagent.

FIG. 5 illustrates the detection of an LC-CRP complex of a patient sample tested over ten days utilizing a Slope-1 optical transmittance measurement of an APTT assay (signal/threshold) utilizing different coagulation instruments with a Control (no anionic surfactant added to the APTT reagent) and 0.005% w/v of anionic surfactant added to the APTT reagent.

FIG. 6 illustrates the analysis of a patient sample over the period of six days utilizing a Slope-1 optical transmittance measurement of an APTT assay (signal/threshold) utilizing different coagulation instruments with a Control (no anionic surfactant added to APTT reagent) and 0.005% w/v of anionic surfactant added to APTT reagent.

FIG. 7 illustrates the lipoprotein profile and CRP levels of an intensive care unit (ICU) patient during the span of twelve days as compared to the lipoprotein profile of two normal patients.

### DETAILED DESCRIPTION

C-Reactive Protein (CRP) is an acute phase plasma protein that forms a complex with lipoproteins in the presence of a divalent cation in vitro when certain medical conditions are present. The measurement of a patient CRP level alone is not necessarily equivalent to the measurement of an LC-CRP complex formed in the presence of a divalent cation. Whether CRP forms a complex with a lipoprotein depends upon the characteristics of the endogenous lipoprotein profile in a patient blood sample.

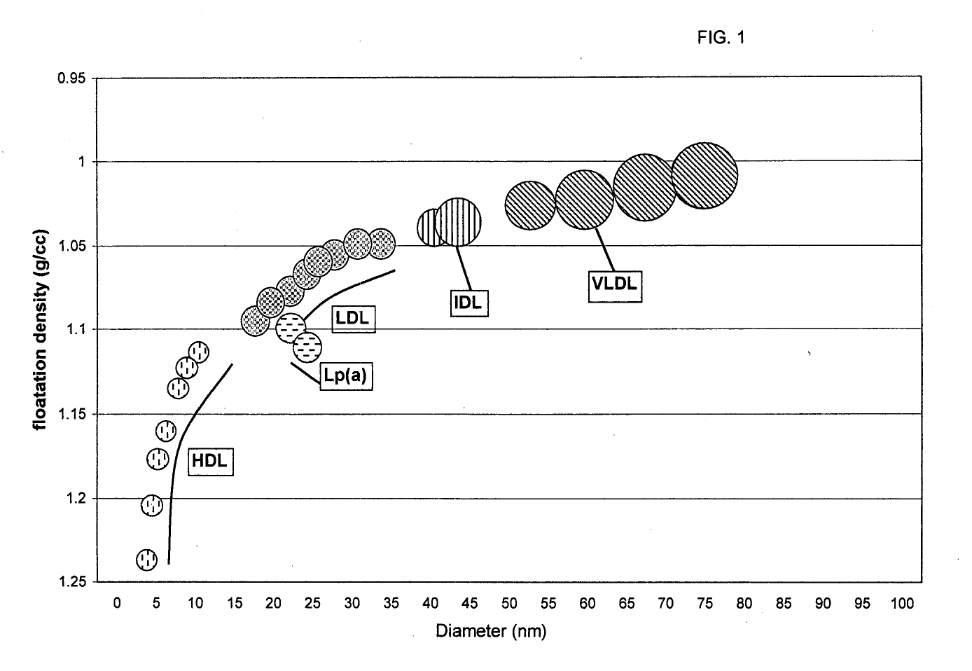

The endogenous lipoproteins that form the LC-CRP complex may be composed of lipoprotein fractions of differing density, diameter and structural apolipoproteins, as illustrated in FIG. 1. HDL is an α-lipoprotein with a density (g/ml) ranging from approximately 1.063 g/ml to approximately 1.21 g/ml and a diameter from approximately 5 nm to approximately 15 nm. LDL is a β-lipoprotein with a density ranging from approximately 1.019 g/ml to approximately 1.063 g/ml and a diameter ranging from approximately 20 nm to approximately 40 nm. The Lp(a) has a density ranging from approximately 1.06 nm to approximately 1.10 nm and a diameter ranging from approximately 20 nm to approximately 40 nm. The IDL is a β-lipoprotein with a density ranging from approximately 1.006g/ml to 1.019 g/ml and a diameter ranging from approximately 20 nm to approximately 35 nm. The VLDL is also a β-lipoprotein with a density ranging from approximately 1.006 g/ml to approximately 0.95 g/ml and a diameter ranging from approximately 30 nm to approximately 90 nm. LDL, IDL, and VLDL all share the common structural apolipoprotein apo-β-100, whereas HDL has A-I and A-II as the primary apolipoproteins. Typically, the in vitro LC-CRP complex is formed primarily with β-lipoproteins (LDL, IDL and VLDL).

In accordance with the invention, it was found that the smaller β-lipoproteins (LDL and IDL) do not consistently form a detectable complex in vitro with CRP. The VLDL-CRP complex was found to be more easily detected. According to the invention, the fact that LDL-CRP and IDL-CRP were not consistently detected with standard methods hindered the overall effectiveness of using LC-CRP as a biomarker. It is diagnostically important in that all types and levels of the endogenous β-lipoproteins and endogenous CRP are measured when a critical ratio of lipoproteins to CRP is reached in a patient blood sample.

According to the invention, the binding between the divalent cation, CRP and the lipoprotein fraction is improved when a surface-active agent is utilized. This has been found to be particularly evident in lipoprotein profiles comprising primarily of smaller, denser lipoproteins (LDL, IDL or a mixture thereof, particularly LDL) or limiting amounts of VLDL.

Any suitable surface-active agent (such as surfactants, detergents, dextrans, polyethoxyglycols and the like) may be utilized in the invention so long as the agent facilitates the LC-CRP complex precipitation. Examples of a preferred group of surface-active agents are anionic surfactants including blends of phosphorylated ethyoxylates, sodium dioctyl sulfocsuccinate, and mixtures thereof. Particularly preferred are fatty acid substituted with polyoxyethylene (POE) compounds, more preferably POE alkyl phenol phosphate. The most preferred surfactant is poly (oxy-1, 2 ethanediyl, alpha-(nonylphenyl)-omego-hydroxy). phosphate, currently marketed as Chemfac® PC099 (Chemax, Inc., PO Box 6067, Greenville, S.C. 29606).

The surface-active agent utilized in the reagent should be used in an amount sufficient to improve the binding of the complex. It may be added in an aqueous solution or as otherwise suitable for the assay design. Preferably, the range utilized is from about 0.0001% surfactant to about 2% w/v, more preferably from 0.001 to 0.02% w/v, and most preferably from 0.002 to 0.01% w/v of the final solution.

While not wishing to be bound by theory, it is believed that the surface-active agent assists the smaller endogenous lipoprotein to form the complex with the endogenous CRP. It is this enhanced complex formation that allows for better detection of the complex.

Any reagent may be used where the sample is combined with a divalent calcium component and the surface-active agent. Suitable reagents among which the surface-active agent may be included comprise clotting assays or screening assays, such as the APTT, prothrombin protein C, fibrinogen, protein S and/or thrombin time. Particularly useful is the APTT assay due to its standard use in coagulation instruments. Alternatively, the complex can be formed and detected utilizing only sample and a divalent cation (preferably calcium) source, referred to herein as an LC-CRP assay.

The patient blood sample that may be tested includes plasma, whole blood, serum and the like. Acceptable forms of the divalent cation that may be used include metal ions, preferably divalent metal ions selected from the group consisting of calcium, magnesium, manganese, cobalt, iron or barium. Most preferably the divalent cation source is calcium, most preferably in the form of calcium chloride. The divalent cation combined with the blood sample should be in an amount sufficient to activate the CRP molecule. Preferably the amount of divalent cation used is from about 3 mM to about 100 mM.

The invention is particularly useful for patient samples having a VLDL-poor profile during an acute phase reaction. The diagnostic utility of the LC-CRP complex is thus improved because complexes comprised primarily of LDL-CRP, IDL-CRP or a combination thereof can be better detected. Preferably the inventive method is utilized in critically ill patient populations with LDL and/or IDL present in an amount greater than 50% of the total β-lipoprotein fraction. The utility is particularly noticeable in critically ill patients having a preponderance of endogenous LDL.

The method of measuring or otherwise detecting LC-CRP complex may be accomplished by any means known to those skilled in the art, by qualitative as well as quantitative methods. For example, the turbidity of the complex may be detected mechanically (including acoustically), electrically or optically (using absorbance or transmittance) either directly or indirectly. Additionally, the LC-CRP complex may be detected or measured by other means such as latex agglutination or gold sol assay, ligand assay, protein binding assay or immunoassay.

In a preferred embodiment, the LC-CRP complex is detected by measuring the turbidity changes prior to fibrin polymerization during the clotting reaction. The detection means is compatible with automated instruments designed to access coagulation. Alternatively, it is not necessary to use a reagent that promotes the formation of a clot in the sample. For example, serum may be used to determine whether the sample will form an in vitro LC-CRP complex.

As has been associated in the prior art, the LC-CRP complex is an indicator of critically ill patients and provides biomarker of those patients having hemostatic dysfunction and severe infection disorders, such as SIRS, sepsis, sepsis-related morbidity or DIC. The monitoring or detection of the LC-CRP may be used in conjunction with other known criteria used in diagnosing patients with these disorders, including an assessment of the underlying medical condition, clinical condition and laboratory results (platelet count, procalcitonin (PCT), fibrinogen, cytokines, acute phase proteins, D-dimer and so on). Such criteria are defined in accordance with the Japanese Ministry of Health and Welfare proposed guideline, now the basis of the International Society of Thrombosis and Hemostasis Standardization Subcommittee definition (Kobayashi N, et al., Bibl. Haematol. (1987) 49:265-275) or the APACHE II scoring system (Knaus W A, et al., Crit. Care Med. (1985) 13: 818-829). The inventive method may effectively be combined with another diagnostic test, particularly coagulation markers, such as d-Dimer, Protein C, AT-III, prothrombin (PT), fibrinogen, Protein S, PAI-I and so on.

In a preferred embodiment, the LC-CRP complex is detected by observation of a biphasic waveform. This provides a quick determination and may be an efficient tool in risk stratification as well as diagnosis and monitoring. Biphasic waveforms may be identified and measured by appropriate methods known to those skilled in the art utilizing coagulation assays and means for measuring the precoagulation phase (slope-1) over a time to provide a profile with determined normal and abnormal parameters. (See, e.g., Toh, et al., Intensive Care Med. (2003) 29:55-61). The waveform measurement used in the diagnosis may be one or more quantitative or qualitative measurements. Preferably, an activated partial thromboplastin time (APTT) assay or an LC-CRP assay is used with an optical transmittance or absorbance coagulation analyser, more preferably APTT, utilizing a single point analysis (threshold flag either indicating the presence or absence of the LC-CRP complex), or several points or the entire waveform, as known by those skilled in the art. For example, in a normal patient sample, the waveform at 580 nm has a sigmoidal waveform pattern and is generally characterized by an initial 100% light transmittance phase prior to formal clot formation. In contrast, critically ill patients having samples that form an LC-CRP complex will have a waveform at 580 nm that shows an immediate and progressive fall in light transmittance in the pre-clotting phase that affects the early part of the curve to produce a biphasic profile.

Unexpectedly, the detection of the LC-CRP complex formation from an abnormal patient sample is improved with the inventive method. For critically ill patients having primarily LDL and/or IDL β-lipoprotein profiles, the inventive method may improve detection anywhere from two-fold to in excess of ten-fold, more preferably from at least two-fold to approximately five-fold, most preferably at least two-fold, as compared to methods not using the inventive method. The improvement thereby provides a more specific biomarker for patient populations having greater organ dysfunction, organ failure, ICU length of stay, mortality and sepsis incidence as well as overt DIC (collectively referred to herein as having hemostatic dysfunction, sepsis-related morbidity or severe infection).

The following non-limiting examples illustrate the invention.

### EXAMPLES

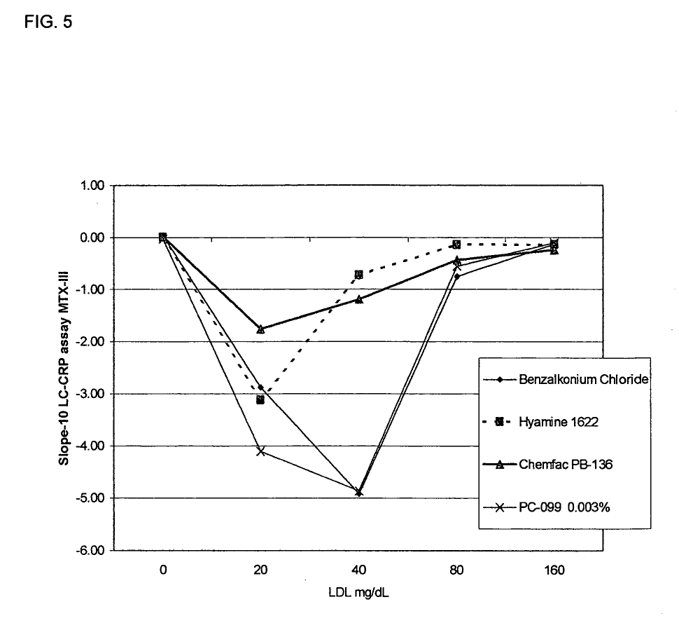

The turbidity of samples was measured utilizing optical transmittance waveform analysis on automated coagulation instruments (“Biphasic Waveform Analysis”), as known to those skilled in the art. Any turbidity changes were measured beginning from 2 to 8 seconds after the reagent was contacted with the sample. The light transmission was then optically monitored and a linear rate taken until approximately 25 seconds had passed (pre-coagulation phase). Measurements were at 405 nm (mODs/min−slope-1). As reflected in FIGS. 5-7, threshold values were established based on the responses from specimens of 40 normal, healthy donors, with any signal/threshold value “1” or greater indicating the detection of an LC-CRP complex. Either an MDA automated coagulation instrument or a MTX automated coagulation instrument (both available from bioMerieux, Inc., Durham, N.C.) was used to take the optical measurement.

Blood samples were collected in 0.105 M trisodium citrate at a ratio of 1 part citrate to 9 parts whole blood. Reaction mixtures for Examples 3-6 were prepared such that a ⅓ amount of APTT reagents was combined with ⅓ plasma sample and ⅓ calcium chloride 20 mM concentration (reacting at 6.67 mM). An anionic surfactant, a fatty acid substituted with a polyoxyethylene compound Chemfac® PC099 (Chemax, Inc., PO Box 6067, Greenville, S.C. 29606), was added in varying amounts, as specially described in each example.

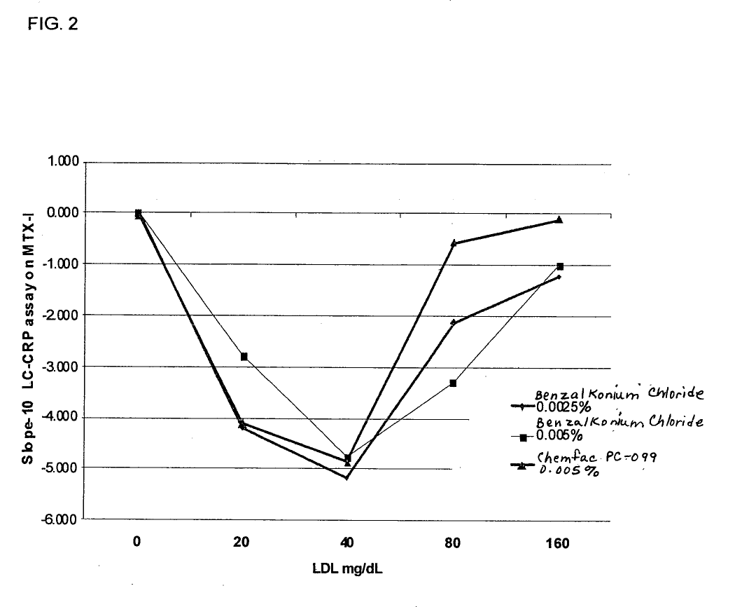

The MDA uses a commercially available probe cleaner containing the anionic surfactant Chemfac PC099. The MDA probe cleaner is as described in U.S. Pat. No. 5,749,976. As set forth in FIGS. 2-4, the testing for the surfactant titration was performed on an MTX instrument, which did not utilize probe cleaning solution containing the surfactant PC099. As set forth in FIGS. 5-7, the MDA Prior Art results represent effects from potential residual Chemfac PC099 present in the assay as introduced by the probe cleaner. For the “MDA 0.005%” Inventive Method in FIGS. 5-7, the MDA was rinsed five times with a commercially available product, MDA Imidazol Buffer, to ensure that any potential residual Chemfac PC099 was removed from the system. The addition of PC099 at 0.005% was accomplished by adding the PC099 directly to the Calcium Chloride reagent. The MTX run (MTX 0.005%) had no residual anionic surfactant because the MTX did not utilize probe cleaner containing Chemfac PC099. As with the MDA, the PC099 was added directly to the calcium chloride reagent at 0.005%.

### Example 1

LDL, VLDL and Normal Plasma Pools (NPP) lipoprotein fractions were prepared in four concentrations levels of 20 mg/dL, 40 mg/dL, 80 mg/dL, and 160 mg/dL. These lipoprotein preparations were then combined with 300 mg/L of CRP with an APTT reagent system containing 6.67 mM calcium chloride (PLATELIN-L, available from bioMérieux, Inc., Durham, N.C.) and 5 different ratios of aqueous solutions containing differing amounts of the anionic surfactant Chemfac PC099. The amounts of anionic surfactants utilized were 0%, 0.0004%, 0.0008%, 0.0015%, 0.0019% and 0.003% w/v.

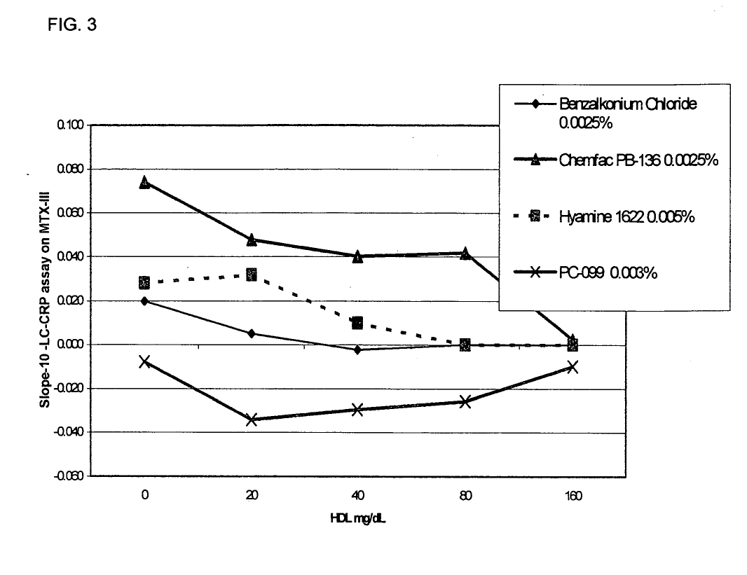

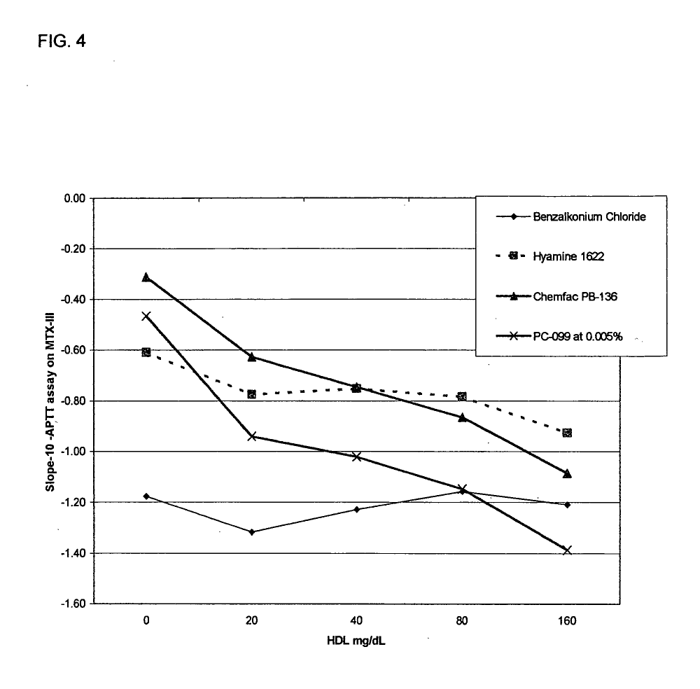

A Biphasic Waveform Analysis was conducted on an MTX. Results for LDL are provided in FIG. 2. Results for VLDL are provided in FIG. 3. Results for NPP are provided in FIG. 4. The results illustrate the effectiveness of the anionic surfactant in controlled amounts facilitating the detection of the LC-CRP complex, particularly for the LDL fraction.

### Example 2

Eight sets of samples were selected from patients adjudicated with a diagnosis of sepsis. The patient adjudication was provided by a panel of physicians utilizing patient history, clinical symptoms, physical examination and laboratory findings. The specimens of the patients had been subject to a quantitative procalcitonin (PCT) test, a quantitative CRP test, and a total cholesterol test using standard, available clinical laboratory tests. APTT testing was also performed on these sets of samples. With the MDA Prior Art Method (no added surfactant) condition, potential residual Chemfac PC099 may have been present in the reaction arising from the use of on-board probe cleaner. With the Inventive Method (MDA 0.005% w/v surfactant and MTX 0.005% w/v surfactant), Chemfac PC099 was added to the CaCl₂ reagent of the APTT reagent system. The reaction mixture was ⅓ APTT reagent; ⅓ sample; and ⅓ calcium chloride (and anionic surfactant if used).

Results from the tests are provided in Table I below, where a “+” PCT value was a reading of greater than 1.5 ng/ml. As reflected in Table I, all eight patient samples had a negative (normal) waveform (“−”) on the MDA automated coagulation instrument, thus indicating no in vitro formation of the LC-CRP complex detected during the pre-coagulation phase for the Prior Art Method. When the samples were retested using 0.005% w/v anionic surfactant (Chemfac PC099), seven of the eight samples showed a positive biphasic waveform (“+”) thus indicating the presence of an LC-CRP complex on both the MDA and MTX coagulation systems. CRP levels and total cholesterol levels were also tested for all patient samples. Results are summarized in Table I below.

TABLE I

| | | | INVENTIVE | INVENTIVE | | |
|---|---|---|---|---|---|---|
| | | PRIOR ART | METHOD MDA | METHOD MTX | | |
| | | METHOD MDA | 0.005% w/v | 0.005% w/v | | Total |
| | | 0.0% Surfactant | Surfactant | Surfactant | | Cholesterol |
| Sample | | Added Biphasic | Added Biphasic | Added Biphasic | Max. CRP | Range |
| ID | PCT | Waveform Result | Waveform Result | Waveform Result | mg/L | mM |
| 1 | + | − | + | + | 170 | 3.2-3.8 |
| 2 | + | − | + | + | 146 | 3.9-4.8 |
| 3 | + | − | + | + | 181 | 1.1-1.4 |
| 4 | + | − | + | + | 128 | 2.1-2.5 |
| 5 | + | − | − | − | 89 | 1.5-3.3 |
| 6 | + | − | + | + | 142 | 1.9-3.0 |
| 7 | + | − | + | + | 149 | 1.2-2.5 |
| 8 | + | − | + | + | 226 | 2.1-4.1 |

### Example 3

Blood was drawn from a critically ill patient in the Intensive Care Unit each day for a period of 10 days. The patient was independently determined to have an infection using clinical evidence, as adjudicated by a panel of physicians utilizing patient history, clinical symptoms, physical examination and laboratory findings. The patient blood samples were tested for the presence of LC-CRP complex using the Biphasic Waveform Analysis with three methods, (1) MDA Prior Art (No (0%) surfactant added), (2) MDA 0.005% w/v Chemfac PC-099 (Inventive Method); and (3) MTX 0.005% w/v Chemfac PC-099 (Inventive Method). With the MDA Prior Art method, potential residual Chemfac PC-099 may have been present arising from the use of on-board probe cleaner.

Results from the Biphasic Waveform Analysis are graphically displayed in FIG. 5. As indicated in the graph, there was over a five-fold improvement for days 1 and 2 in detecting the LC-CRP complex utilizing the inventive method. As reflected in the graph, the Inventive Method enabled a more sensitive quantitative detection (improved dynamic range) of the LC-CRP complex on both the MDA and MTX coagulation systems.

### Example 4

Blood was drawn from a critically ill patient in the Intensive Care Unit each day for a period of 6 days. The patient was independently determined to have an infection using clinical evidence, as adjudicated by a panel of physicians utilizing patient history, clinical symptoms, physical examination and laboratory findings. The patient blood samples were tested for the presence of LC-CRP complex using the Biphasic Waveform Analysis with three methods, (1) MDA 0.0% w/v (Prior Art Method), (2) MDA 0.005% w/v Chemfac PC-099 (Inventive Method); and (3) MTX 0.005% w/v Chemfac PC-099 (Inventive Method). With the MDA Prior Art Method (0.0% w/v surfactant added), potential residual Chemfac PC-099 may have been present arising from the use of on-board probe cleaner.

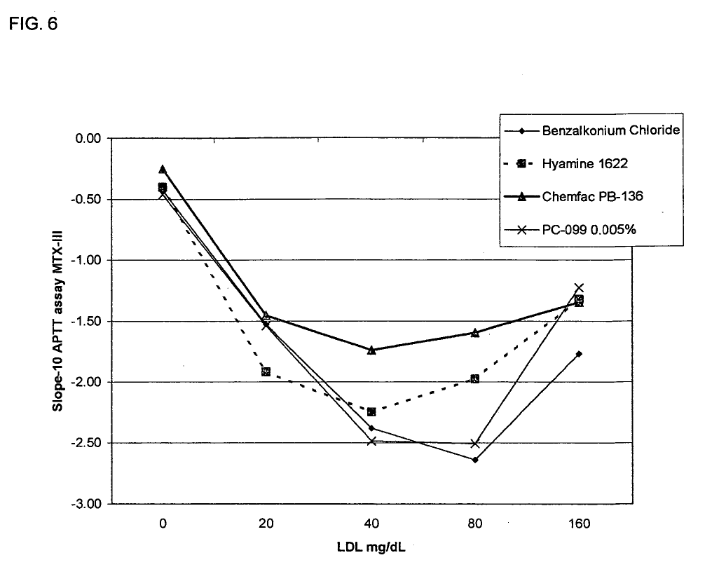

Results from the Biphasic Waveform Analysis are graphically displayed in FIG. 6. As indicated in the graph, the MDA Prior Art Method showed no LC-CRP complex (threshold of 1 not met). For days 2-5, the Inventive Method showed the presence of an LC-CRP complex on both the MDA and MTX coagulation systems.

### Example 5

Blood was drawn from a critically ill patient in the Intensive Care Unit each day for a period of 12 days. The patient was independently determined to have an infection using clinical evidence, as adjudicated by a panel of physicians utilizing patient history, clinical symptoms, physical exam and laboratory findings. The patient was diagnosed with DIC on days 3-4 utilizing the criteria defined in accordance with the Japanese Ministry of Health and Welfare proposed guidelines, now the basis of the International Society of Thrombosis and Hemostasis Standardization Subcommittee definition Kobayashi N., et al., Bibl. Haematol. (1987) 49:265-275).

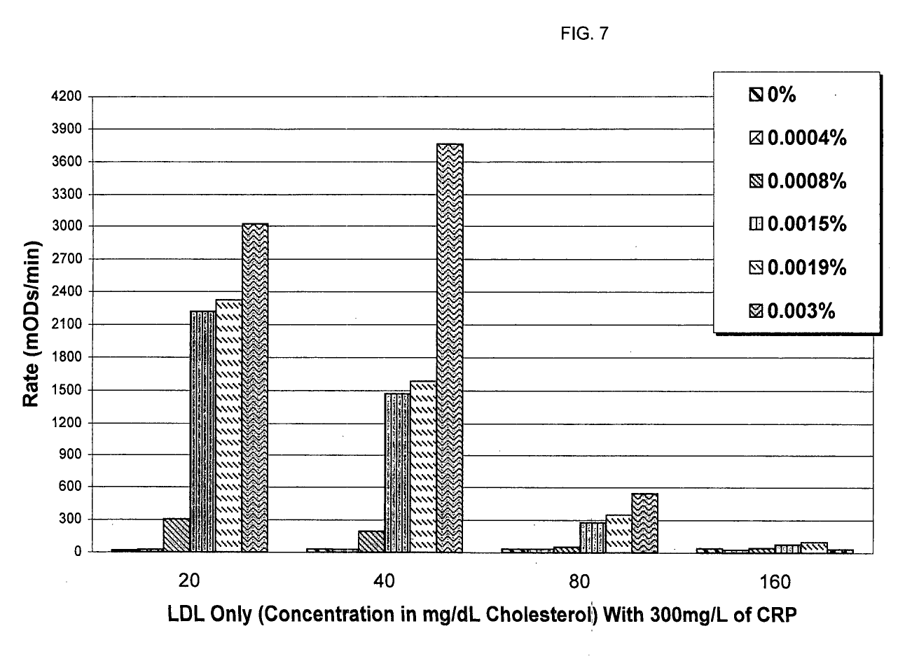

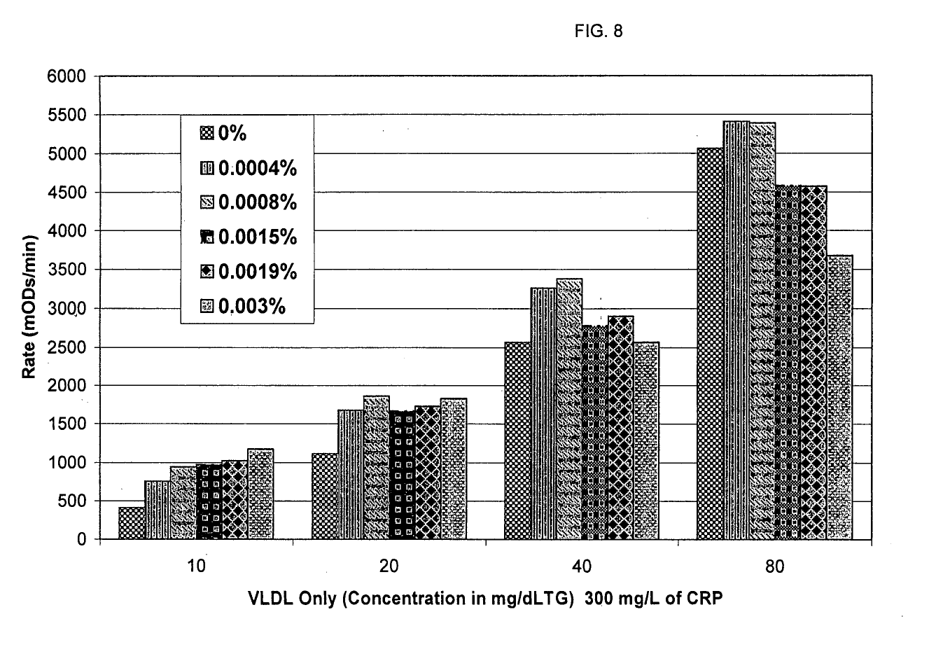

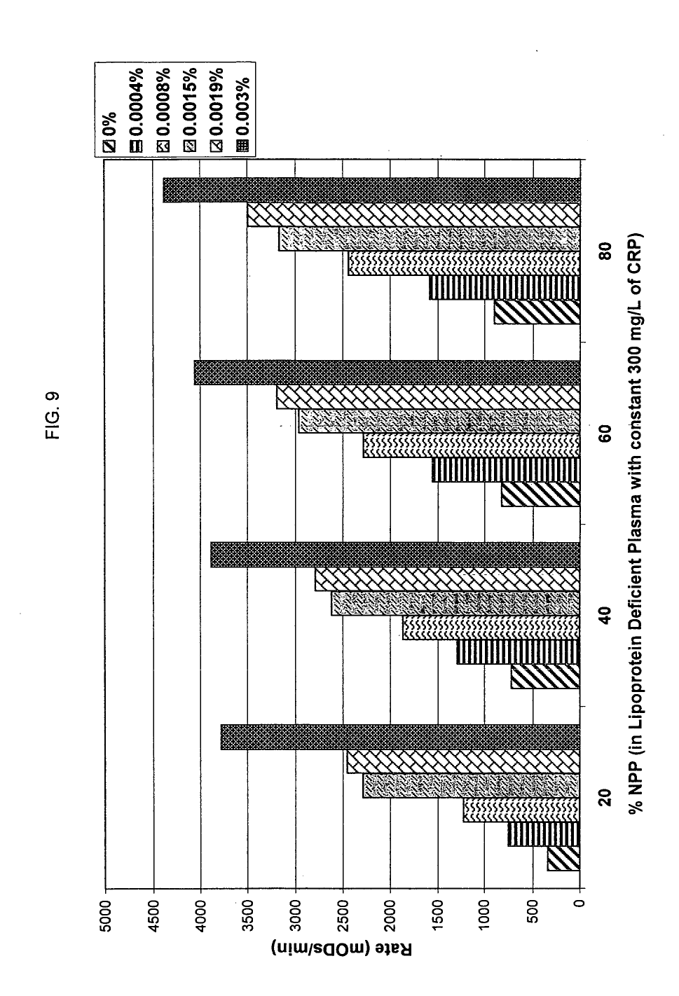

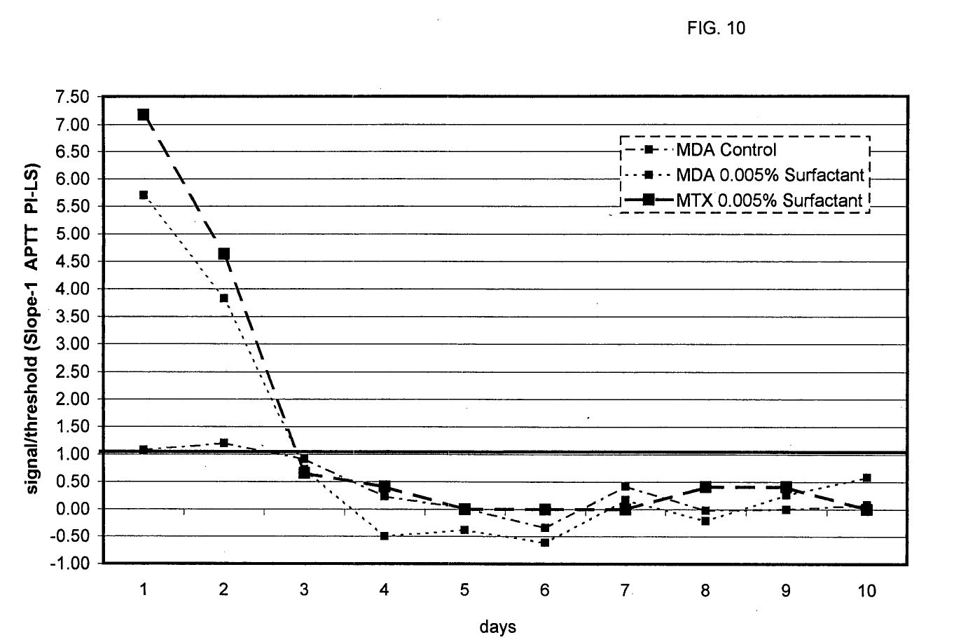

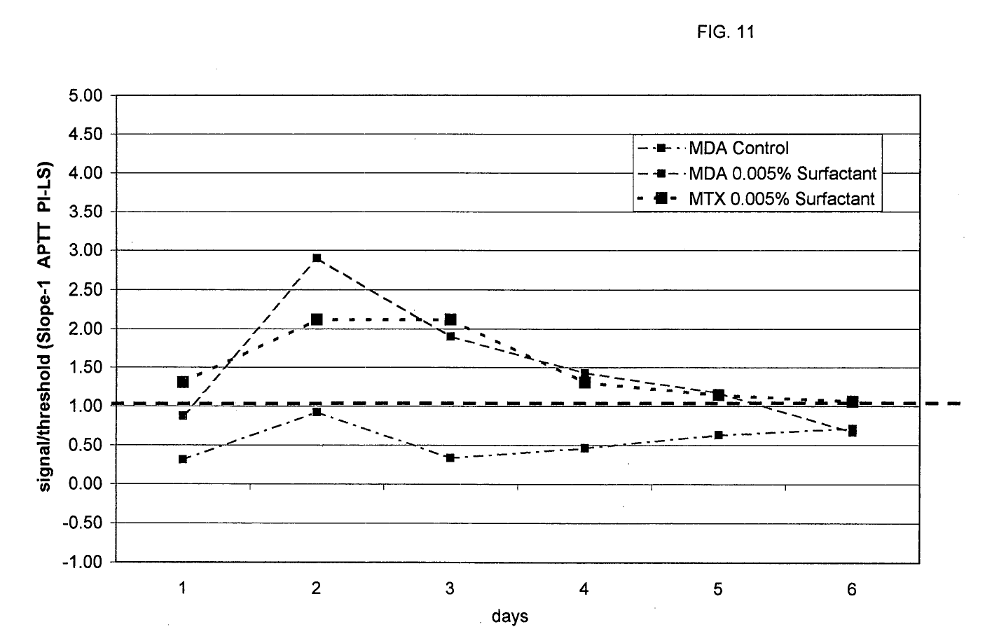

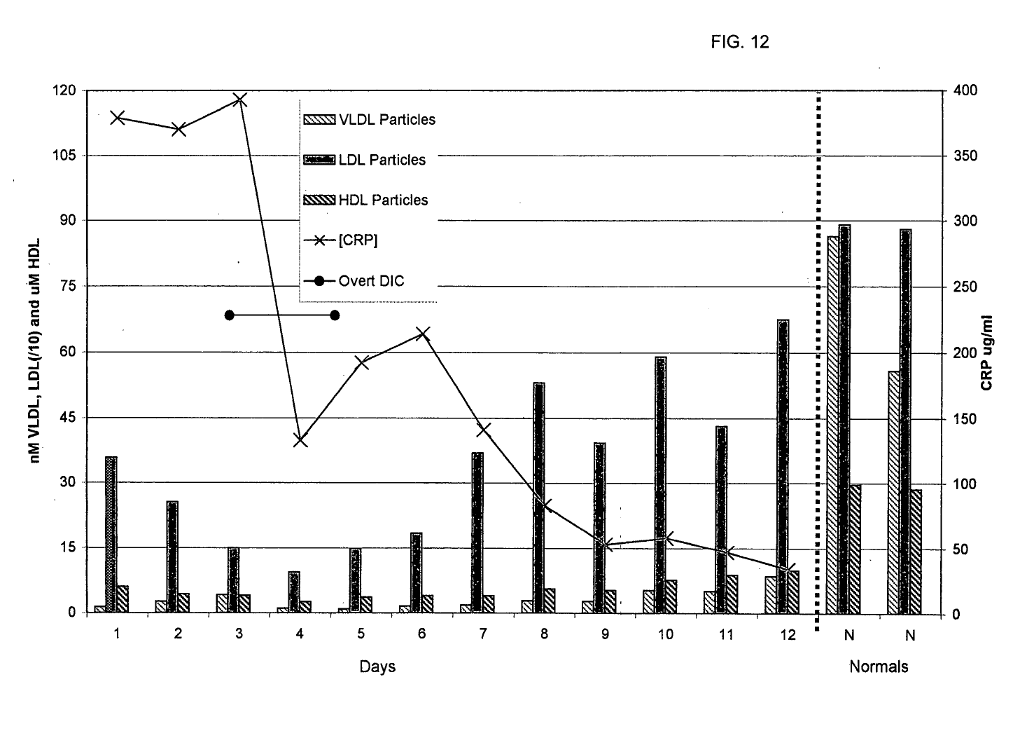

A Biphasic Waveform Analysis (Prior Art Method) was conducted on the patient samples for each day. For days 1-6, an abnormal waveform was measured, thus indicating the presence of the LC-CRP complex. A Nuclear magnetic Resonance (NMR) analysis of the lipoprotein profile was conducted for each sample collection. Additionally, a quantitative test was run to determine the CRP level. Results are shown in FIG. 7, as compared with two normal patient samples. This example showed that the lipoprotein fraction differed over the course of the twelve days, thus supporting the need for detecting not only the VLDL but also the LDL fraction.

### Example 6

Blood samples taken from 104 critically ill patients admitted to the Intensive Care Unit were tested under the Biphasic Waveform Analysis. The patient samples were tested using either Prior Art Method (⅓ of APTT reagent; ⅓ of 6.67 mM calcium chloride; ⅓ of plasma sample) or the Inventive Method (⅓ of APTT reagent; ⅓ of 6.67 mM calcium chloride with 0.005% w/v Chemfac PC099; ⅓ of plasma sample). The Prior Art Method potentially contained residual anionic surfactant because the measurement was taken using an MDA that used on-board MDA probe cleaner. With the Inventive Method, care was taken to remove any potential PC099 surfactant residue by eliminating all on-board MDA probe cleaner. In the Inventive Method the anionic surfactant was added to the calcium chloride reagent at 0.005% w/v. The abnormal Waveform threshold was established through the analysis of normal donor waveforms. For graphical purposes the data are shown as the patient Waveform measurement divided by the threshold. Any sample with a signal to threshold ratio of 1 or greater was designated as a positive Waveform. A procalcitonin (PCT) test was also run on all 104 samples. Any patient with a minimum PCT reading of 1.5 ng/ml or greater was designated with a “+” PCT.

Results are provided in Table II below. As shown, the Inventive Method showed a sensitivity rate of 93.3% as compared to a sensitivity rate of 44.4% for the Prior Art Method.

TABLE II
Prior Art Method
(MDA with 0% anionic surfactant added to APTT reagent)

| | Sepsis+ | Sepsis− | Total Patients |
|---|---|---|---|
| Waveform+ | 20 patients | 35 patients | 55 |
| Waveform− | 25 patients | 24 patients | 49 |
| TOTAL | 45 | 59 | 104 |

SENSITIVITY = 44.4%
SPECIFICITY = 40.7%
ACCURACY = 42.7%
PPV 36%
NPV 49%

Inventive Method
(MDA with 0.005% w/v anionic surfactant added to APTT reagent)

| | Sepsis+ | Sepsis− | Total Patients |
|---|---|---|---|
| Waveform+ | 42 patients | 43 patients | 85 |
| Waveform− | 3 patients | 16 patients | 19 |
| TOTAL | 45 | 59 | 104 |

SENSITIVITY = 93.3%
SPECIFICITY = 27.1%
ACCURACY = 56%
PPV 49.4%
NPV 84.2%

Procalcitonin (PCT)

| | Sepsis+ | Sepsis− | Total Patients |
|---|---|---|---|
| PCT 1.5+ | 36 patients | 36 patients | 72 |
| PCT 1.5− | 9 patients | 23 patients | 32 |
| TOTAL | 45 | 59 | 104 |

SENSITIVITY = 80.0%
SPECIFICITY = 39.0%
ACCURACY = 57%
PPV 50.0%
NPV 71.9%

That which is claimed is:

1. A method for diagnosis and monitoring a hemostatic dysfunction, sepsis-related morbidity or severe infection, said method comprising (a) obtaining a patient sample; (b) combining said sample with a reagent comprising a divalent cation and an effective amount of a surface active agent to form a reaction mixture; and (c) examining said reaction mixture to determine whether an LC-CRP complex is formed to diagnose or monitor patients having hemostatic dysfunction, sepsis-related morbidity or severe infection.

2. A method according to claim 1 wherein said surface active agent is an anionic surfactant.

3. A method according to claim 2 wherein said anionic surfactant is selected from the group consisting of blends of phosphorylated ethyoxylates, sodium dioctyl sulfocsuccinate, and mixtures thereof.

4. A method according to claim 3 wherein said anionic surfactant comprises a POE alkyl phenol phosphate present in an amount ranging from about 0.0001% to about 2% w/v.

5. A method according to claim 4 wherein said surfactant consists essentially of poly(oxy-1,2 ethanediyl, alpha-(nonylphenyl)-omego-hydroxy-phosphate) present in an amount ranging from about 0.002% to 0.01% w/v.

6. A method according to 1 wherein said surface active agent comprises poly(oxy-1,2 ethanediyl, alpha-(nonylphenyl)-omego-hydroxy-phosphate).

7. A method according to claim 1 wherein said surface active agent is present in an amount ranging from about 0.0001% to about 2% w/v.

8. A method according to claim 1 wherein said surface active agent is present in an amount ranging from 0.001% to 0.02% w/v.

9. A method according to claim 1 wherein said surface active agent is present in an amount ranging from 0.002% to 0.01% w/v.

10. A method according to claim 1 wherein said method increases the detection of the LC-CRP complex at least about ten-fold.

11. A method according to claim 1 wherein said method increases the detection of the LC-CRP complex at least about five-fold.

12. A method according to claim 1 wherein said method increases the detection of the LC-CRP complex at least about two-fold.

13. A method according to claim 12 wherein said method further comprises detecting said LC-CRP complex by utilizing a waveform analysis for said determination and correlating said determination to one or more known measurements of LC-CRP complex to diagnose or monitor patients.

14. A improved method of diagnosing sepsis or DIC comprising detecting an in vitro formation of an LC-CRP complex formed upon combining a blood sample with a divalent cation, said improvement comprising utilizing a surface active agent present in an effective amount to assist in forming LC-CRP complex comprising IDL, LDL or mixtures thereof or limited VLDL.

15. A method according to claim 14 wherein said LC-CRP complex consists of LDL complexed with CRP.

16. A method for diagnosing hemostatic dysfunction, sepsis-related morbidity or severe infection, said method comprising (a) obtaining a patient sample; (b) combining said sample with reagents comprising a calcium component and an effective amount of surface active agent to form a reaction mixture; (c) subjecting said reaction mixture to a waveform analysis to obtain a normal or biphasic waveform result based on the detection of a LC-CRP complex; and (d) comparing said waveform result in a model to distinguish patients with a diagnosis of hemostatic dysfunction, sepsis-related morbidity or severe infection from normal healthy patients by one or more qualitative or quantitative measurements.

17. A method according to claim 16 wherein said surface active agent is present in an amount ranging from 0.002% to 0.01% w/v and said surfactant improves the detection of the LC-CRP complex by at least about 2 fold for those patients having severe infection, sepsis-related morbidity or hemostatic dysfunction as compared with a divalent cation containing reagent that does not contain an surface active agent; and said measurement is a threshold flag indicating the presence or absence of the LC-CRP complex.

18. A method according to claim 16 wherein said method is used in conjunction with a PCT test in diagnosing a patient with hemostatic dysfunction, sepsis-related morbidity or severe infection.

19. A method according to claim 16 wherein said LC-CRP consists essentially of LDL complexed with CRP.

20. A method according to claim 16 wherein said method is used in conjunction with a coagulation marker.
